(19) United States
(12) Patent Application Publication
(10) Pub. No.: US 2024/0105300 A1
(43) Pub. Date: Mar. 28, 2024

(54) RECEIVING PRESCRIPTION REFILL REQUESTS VIA VOICE AND/OR FREE-TEXT CHAT CONVERSATIONS BETWEEN A PATIENT AND AN AUTOMATED AGENT

(71) Applicant: Providence St. Joseph Health, Seattle, WA (US)

(72) Inventors: Wayne T. Foley, Seattle, WA (US); Reza Ladchartabi, Redwood City, CA (US); Michael Lynch, Toronto (CA); William A. Nagy, Mercer Island, WA (US); Ben Knoechel, Cochrane (CA); Edgardo Gutierrez, Seattle, WA (US); Jonathan Becker, Portland, OR (US); Adam Benjamin Smith-Kipnis, Seattle, WA (US)

(21) Appl. No.: 17/950,867

(22) Filed: Sep. 22, 2022

(51) Int. Cl. G16H 20/10; G16H 10/60; G16H 80/00

(52) U.S. Cl. CPC G16H 20/10; G16H 10/60; G16H 80/00

(57) ABSTRACT

A facility for causing the refill of prescriptions is described. The facility conducts a conversational exchange with a person. From the exchange, the facility discerns (1) an intent of refilling a prescription, and (2) an identification of a prescription to be fulfilled. In response to this discerning, the facility causes a pharmacy refill order to be placed on behalf of the person for the identified prescription.

### BACKGROUND

It is common for physicians to prescribe medications for their patients. After a prescription is generated for a patient by a physician, the patient or the physician conveys the prescription to a pharmacy.

The pharmacy “fills” the prescription by preparing a container that contains the medicine specified by the prescription, in a form, dosage, and quantity specified by the prescription, and delivers it to the patient, such as in person or by mail.

Some prescriptions are written in such a way as to provide for one or more “refills,” such that, after the patient takes all of the medicine in the original container, the patient can obtain one or more similar containers in order to continue taking the medicine. In order to do so, the patient contacts the physician or the pharmacy, such as by going to one of these places in person, or placing a phone call and speaking to a person who works there.

### BRIEF DESCRIPTION OF THE DRAWINGS

FIG. 1 is a data flow diagram depicting the operation of the facility in some embodiments.

FIG. 2 is a block diagram showing some of the components typically incorporated in at least some of the computer systems and other devices on which the facility operates.

FIG. 3 is a flow diagram showing a process performed by the facility in some embodiments in order to conduct a conversation with a user that involves refilling a prescription.

FIG. 4 is a flow diagram showing a process performed by the facility in some embodiments to process a prescription refill request in accordance with user input received by the facility.

### DETAILED DESCRIPTION

The inventors have recognized significant disadvantages in conventional approaches available to patients to request prescription refills. In particular, talking to a person at the pharmacy or the physician's office can be difficult for a patient in many respects: (1) it can take time and effort to travel to a physical location, or determine the correct phone number to call; (2) there are many hours of the day and night when no one is working to speak to the patient, and additional hours when everyone who is working is occupied, requiring the patient to wait to speak to someone, or come back or call back later; (3) it generally takes time for the person to whom the patient speaks to access electronic records about the prescription, which they need in order to determine whether a refill can be ordered and to order it, and this access is subject to manual error; (4) in some cases, the patient may have trouble understanding the speech of the person they speak to, or being understood by them, either in being able to recognize which words are spoken, or in understanding the larger ideas that the speaker is trying to communicate; and (5) conducting a spoken conversation with another person tends to require certain levels of mental, physical, and emotional energy, and each spoken conversation a person conducts can reduce some or all of these levels, making it more difficult to conduct later ones—this can be true both of patients and of workers at pharmacies and physicians' offices.

In response to recognizing these disadvantages, the inventors have conceived and reduced to practice a software and/or hardware facility for receiving prescription refill requests via voice and/or free-text chat conversations between a patient and an automated agent (“the facility”).

The facility conducts a conversation between the patient and an automated agent. In some embodiments, the patient speaks to the automated agent, and the facility generates simulated speech from the agent to the patent. In some embodiments, the patient and the agent send free-form text strings to one another. In some embodiments, these modes are combined, or the conversation is conducted using other similar modalities.

In various embodiments, the patient uses various mechanisms to engage in the conversation, including, for example, a specialized medical assistance application, a voice and/or text chat application, a telephony application, a browser, a plugin to a general-purpose assistant, etc. These can be on a smartphone, a wearable device, a computer system, a POTS handset, a kiosk, or a variety of other devices.

In some embodiments, in the conversation, the facility invites the patient to share their intent for the conversation. Such intents can extend to a broad array of subjects beyond refilling prescriptions. As the conversation proceeds, the facility continues to seek this intent, as well as named entities (“entities”) relevant to the expressed intent, providing context intended to assist the patient in providing this information. For example, when the facility discerns the intent of refilling a prescription, in some embodiments it (1) determines that an entity identifying the particular prescription to be refilled is necessary for fulfilling this intent, and (2) lists the patient's prescriptions to assist the patient in specifying the entity corresponding to the prescription to be refilled. In some embodiments, the facility applies machine learning models to the patient's input in order to discern intent and relevant entities.

Once the facility discerns the intent to refill a prescription and the identification of an entity identifying the prescription to refill, it accesses a record for this prescription, such as one stored for the patient by electronic medical record (“EMR,” or “EHR”) software. Based on the contents of this prescription record, in some embodiments, the facility takes a different path to order the refill. Where the prescription has not expired and a number of refills specified by the physician in writing the prescription has not been reached, the facility proceeds to order the refill from the pharmacy, such as by calling a programmatic ordering interface exposed by the pharmacy; calling an ordering voice-response telephone line operated by the pharmacy with simulated touch-tones and/or computer-generated or manually-recorded voice; calling a live ordering telephone line with computer-generated or manually-recorded voice; or directing a person such as a physician's assistant to call such a live ordering telephone line. Where the prescription has expired or its number of refills has been exhausted, the facility refers a renewal request to the physician who wrote the prescription, or a delegee. If the physician or delegee approves the renewal, the facility proceeds with the order as discussed above. If a problem arises in this process, the facility advises the patient to contact the physician directly.

By operating in some or all of the ways described above, the facility enables the patient to quickly and easily order a prescription refill, using modalities and tools that they find comfortable and effective, at any time that is convenient to them. Also, the facility circumvents many of the categories of errors that can occur in more manual conventional approaches.

Additionally, the facility improves the functioning of computer or other hardware, such as by reducing the dynamic display area, processing, storage, and/or data transmission resources needed to perform a certain task, thereby enabling the task to be permitted by less capable, capacious, and/or expensive hardware devices, and/or be performed with lesser latency, and/or preserving more of the conserved resources for use in performing other tasks. For example, by eliminating the need to order refills by phone, the facility relieves the load imposed on telephony hardware, such as handsets, switches, etc., freeing them up for increased or improved use for other purposes, or permitting them to be replaced with lower-capacity, cheaper alternatives, or even eliminated. Similarly, eliminating the need to order refills in person, the facility relieves the load imposed on vehicles, such as cars, motorcycles, bicycles, buses, taxis, etc., freeing them up for increased or improved use for other purposes, or permitting them to be replaced with lower-capacity, cheaper alternatives, or even eliminated.

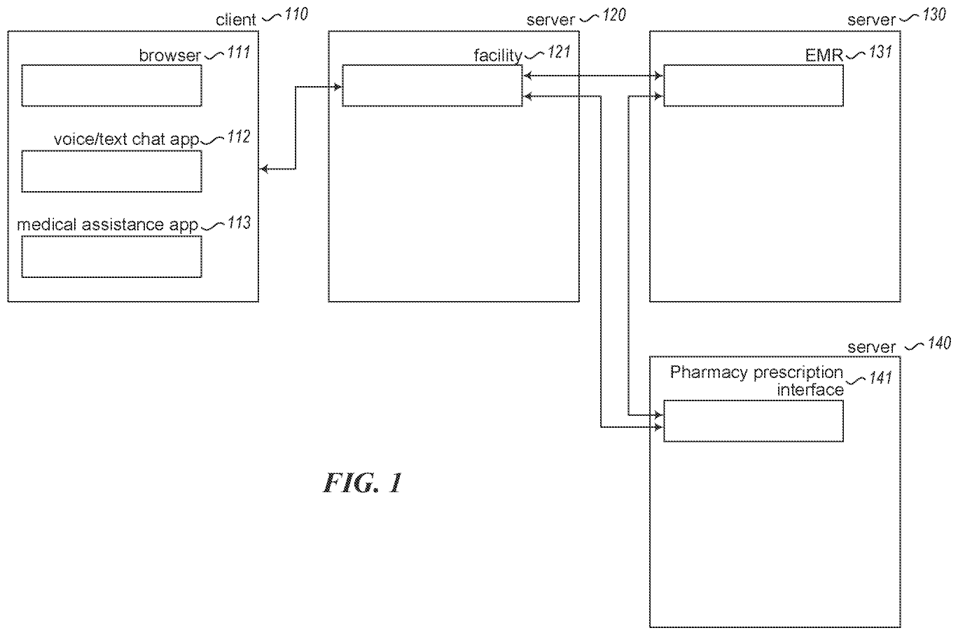

FIG. 1 is a data flow diagram depicting the operation of the facility in some embodiments. Aspects of the facility 121 execute on a server 120. The facility interacts with a client 110 used by a patient or other user. In particular, the user interacts with the facility via one or more programs on the client, such as a browser 111, an application for voice and/or text chat 112, or an application for providing medical assistance 113. The interactions between the client and the facility together constitute a conversational exchange between the user and an automated agent provided by the facility. The facility also interacts with an EMR 131 running on a server 130. In some embodiments, the EMR and/or the facility interact with a pharmacy prescription interface 141 for the user's pharmacy, executing on server 140, in order to submit prescription refill orders on behalf of the user. The interactions between the facility and the EMR can be for any of the following purposes: to retrieve information about the user and particular prescriptions written for the user, including identifying information for the writing physician and people who work with the writing physician, such as their medical assistants; to send messages to the writing physician or associated workers, such as to authorize renewal of the prescription, check for drug interactions with the prescription, manually place orders with the pharmacy, etc.

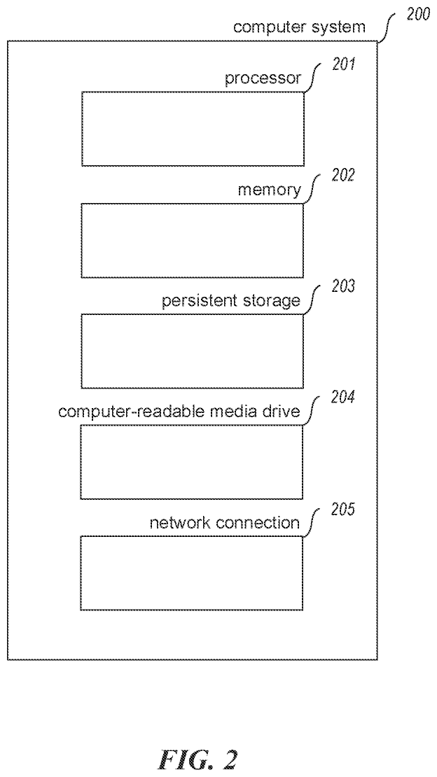

FIG. 2 is a block diagram showing some of the components typically incorporated in at least some of the computer systems and other devices on which the facility operates. In various embodiments, these computer systems and other devices 200 can include server computer systems, cloud computing platforms or virtual machines in other configurations, desktop computer systems, laptop computer systems, netbooks, mobile phones, personal digital assistants, televisions, cameras, automobile computers, electronic media players, etc. In various embodiments, the computer systems and devices include zero or more of each of the following: a processor 201 for executing computer programs and/or training or applying machine learning models, such as a CPU, GPU, TPU, NNP, FPGA, or ASIC; a computer memory 202 for storing programs and data while they are being used, including the facility and associated data, an operating system including a kernel, and device drivers; a persistent storage device 203, such as a hard drive or flash drive for persistently storing programs and data; a computer-readable media drive 204, such as a floppy, CD-ROM, or DVD drive, for reading programs and data stored on a computer-readable medium; and a network connection 205 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

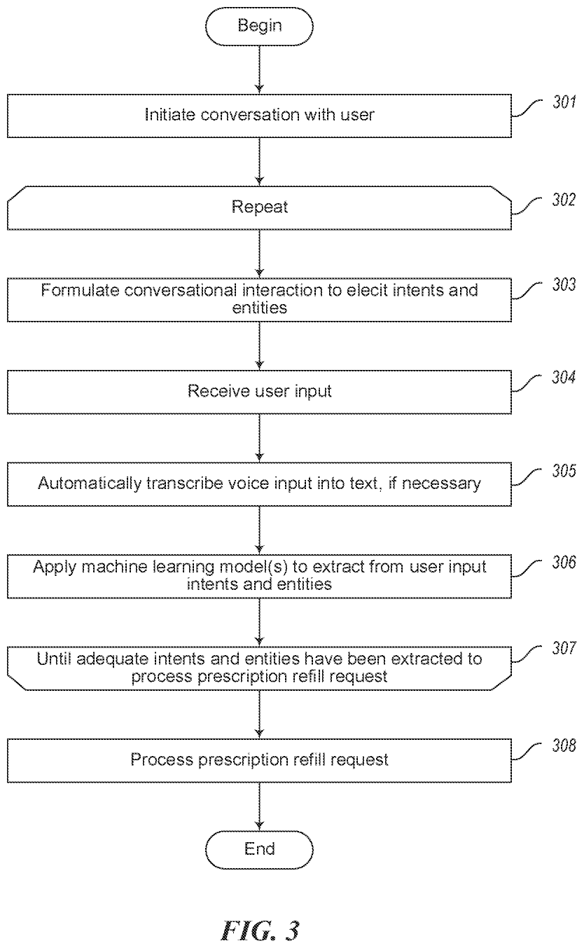

FIG. 3 is a flow diagram showing a process performed by the facility in some embodiments in order to conduct a conversation with a user that involves refilling a prescription. In act 301, the facility initiates the conversation with the user. The facility then repeats acts 302-307 until adequate intents and entities have been extracted from the conversation in order to process a prescription refill request. In act 303, the facility formulates a conversational interaction for the smart assistant intended to elicit intense and associated entities from the user. For example, in some embodiments, the facility formulates and advances the conversational interaction shown below in Table 1. In the tables that follow, interactions from the smart assistant are shown on the left, while interactions from the user are shown on the right. In various embodiments, each party's interactions are provided in a variety of modalities, including voice and text.

TABLE 1

| | |
|---|---|
| Smart Assistant | User |

Hi, I'm Grace, your healthcare assistant.
How can I help you today?

In act 304, the facility receives user input, typically responsive to its last conversational interaction from the smart assistant. In act 305, the facility automatically transcribes user input received in the voice modality to text, if this is necessary for the processing of the voice input by the machine learning models used by the facility, and/or in order to be able to display the transcribed text as part of the visual representation of the conversation. In various embodiments, the facility uses various mechanisms to perform the transcription of act 305, including a variety of transcription engines and/or natural language voice models.

Table 2 below shows a sample first user input received in response to the conversational interaction shown above in Table 1.

TABLE 2

| Smart Assistant | User |
|---|---|
| Hi, I'm Grace, your healthcare assistant. | |
| How can I help you today? | |
| | I'd like to refill a prescription. |

In act 306, the facility applies one or more machine learning models to extract from the user input—in some cases as transcribed in act 304—intents and entities that it contains. In various embodiments, the facility uses a variety of machine learning model types. In some embodiments, the facility uses a transformer-based machine learning model, such as those described in any of the following, each of which is hereby incorporated by reference in its entirety: “BERT” (available at huggingface.co/docs/transformers/model_doc/bert); J. Devlin, M. W. Chang, K. Lee, K. Toutanova, “BERT: Pre-training of Deep Bidirectional Transformers for Language Understanding,” arxiv: 1810.04805 (available at arxiv.org/abs/1810.04805); B. Portelli, “DiLBERT (Disease Language BERT)” (available at huggingface.co/beatrice-portelli/DiLBERT); W. Siblini, M. Challal, C. Pasqual, “Delaying Interaction Layers in Transformer-based Encoders for Efficient Open Domain Question Answering,” arxiv: 2010.08422 (available at arxiv.org/abs/2010.08422); and K. Roitero, B. Portelli, M. H. Popescu and V. D. Mea, “DiLBERT: Cheap Embeddings for Disease Related Medical NLP,” in IEEE Access, vol. 9, pp. 159714-159723, 2021, doi: 10.1109/ACCESS.2021.3131386. (available at ieeexplore.ieee.org/document/9628010). In cases where the present application conflicts with the document incorporated by reference, the present application controls.

In the example conversation shown in Tables 1 and 2, the facility extracts the intent of refilling a prescription. In act 307, if adequate intents and entities have been extracted to be able to process the prescription refill request, then the facility continues in act 308, else the facility continues in act 302 to exchange another pair of interactions.

Continuing the example, in the second iteration of act 303, the facility formulates the additional interaction shown below, in which it confirms the intent to refill a prescription and assists the user in identifying the particular prescription to refill by listing those that are active for the user in the user's EMR record. In some embodiments, the facility calls an API exposed by the EMR in order to retrieve information about active prescriptions. For example, by using the EPIC EMR, the facility calls its GetPrescriptionInfo (2018) API for this purpose.

TABLE 3

| Smart Assistant | User |
|---|---|
| Hi, I'm Grace, your healthcare assistant. | |
| How can I help you today? | |
| | I'd like to refill |
| | a prescription. |
| I see you have the following active prescriptions; | |
| which would you like to refill? | |
| Amoxicillin | |
| Metformin | |
| Naproxen | |
| | Metformin |
| Thank you. I have checked to ensure that | |
| Metformin can be refilled now. I will proceed | |
| to place your refill order. | |

Table 3 further shows that, in the second iteration of act 304, the facility receives user input selecting a particular one of these prescriptions, “Metformin,” for refilling. The facility further confirms the selection, and indicates that it will place the refill request order, as discussed below.

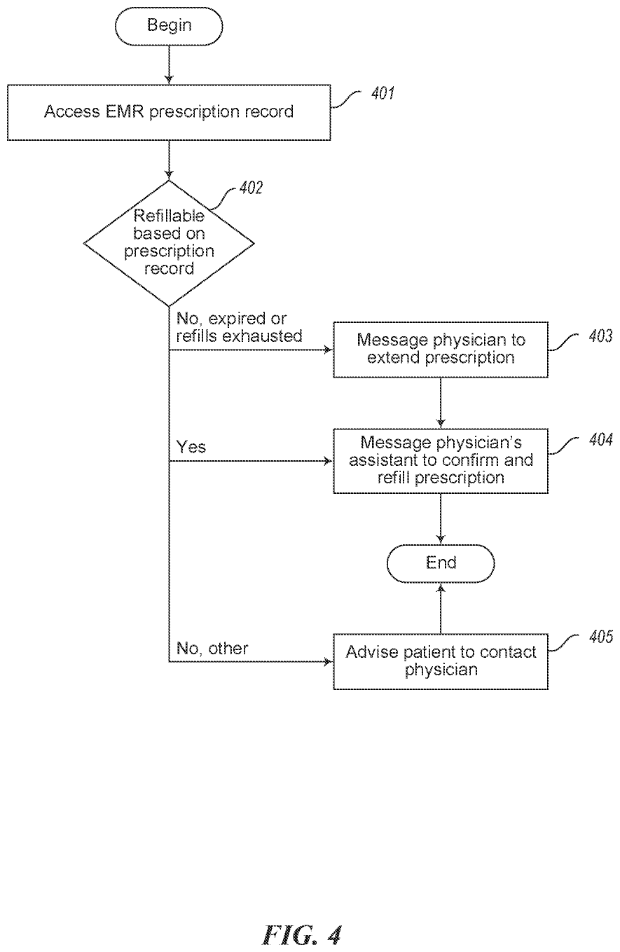

In act 308, the facility processes a prescription refill request for the prescription corresponding to the entity extracted from user input. Additional details of act 308 are discussed below in connection with FIG. 4. After act 308, this process concludes.

Those skilled in the art will appreciate that the acts shown in FIG. 3 and in each of the flow diagrams discussed below may be altered in a variety of ways. For example, the order of the acts may be rearranged; some acts may be performed in parallel; shown acts may be omitted, or other acts may be included; a shown act may be divided into subacts, or multiple shown acts may be combined into a single act, etc.

FIG. 4 is a flow diagram showing a process performed by the facility in some embodiments to process a prescription refill request in accordance with user input received by the facility. In act 401, the facility accesses the EMR prescription record for the selected prescription, such as by calling an API exposed by the EMR for this purpose, such as the GetPrescriptionlnfo (2018) API exposed by EPIC EMR. In act 402, the facility branches based upon whether the prescription is in a refillable state based upon the prescription record accessed in act 401: if the prescription is not immediately refillable because it has expired, or the specified number of refills have already been used, then the facility continues in act 403; if the prescription is refillable, then the facility continues in act 404; and if the prescription is not presently refillable for a reason other than expiration or refills exhausted, then the facility continues in act 405. In act 403, the facility causes a message to be sent to the writing physician identified for the prescription in the accessed EMR prescription record soliciting the physician to extend the prescription, such as by establishing a new, future expiration date, and/or specifying a larger number of refills. After act 403, the facility continues in act 404. In act 404, the facility causes a message to be sent to a physician's assistant to confirm that refill is appropriate, such as by checking for possible drug interactions involving the prescription's drug, and refill the prescription. In some embodiments, the facility causes messages to be sent in act 403 and 404 by calling an API exposed by the EMR for this purpose. For example, when using the EPIC EMR, the facility calls a SendMessage (2013) API exposed by this EMR, in each case specifying a recipient ID determined by the facility for the appropriate recipient, such as reading it from the EMR prescription record accessed in act 401, or reading it from elsewhere in the EMR, such as a patient record for the user. In some embodiments, for the message to the physician, for example, the facility obtains the needed recipient identifier for the physician by calling a GetPCP (2019) API exposed by the EMR. In some embodiments (not shown), in place of act 404, or in addition to act 404, the facility automatically causes a refill prescription order to be placed at the pharmacy, such as by calling an API of the EMR that in turn programmatically places a prescription refill order with the pharmacy; directly calling an API exposed by the pharmacy, or automatically interacting with a system exposed by the pharmacy—such as a voice response telephone system—intended for manual use to order prescription refills, etc. After act 404, this process concludes. In act 405, because the prescription is not immediately refillable and isn't subject to being made refillable by the most typical interactions with the writing physician to permit the physician to obtain additional information from the user that may be useful, the facility advices the patient to contact the physician, such as by including a conversational interaction to that effect in the conversation with the user, sending the user a separate message of any of a variety of types, etc. After act 405, this process concludes.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method in a computing system, comprising:
receiving first natural language input from a user;
after receiving the first natural language input from the user:
subjecting the first natural language input to natural language processing techniques that discern in the first natural language input an intent to refill a prescription;
providing first natural language output to the user acknowledging the intent to refill a prescription;
receiving second natural language input from the user;
after receiving the second natural language input from the user:
subjecting the second natural language input to natural language processing techniques that discern in the second natural language input a named entity corresponding to a prescription to be refilled;
providing second natural language output to the user acknowledging the discerned named entity; and
causing a pharmacy refill order to be placed on behalf of the user for the prescription to which the discerned named entity corresponds.

2. The method of claim 1 wherein the natural language processing techniques to which the first and second natural language input are subjected are one or more machine learning language models trained to predict (1) intents and (2) named entities.

3. The method of claim 2 wherein the machine learning language model is a deep bidirectional transformer for language understanding.

4. The method of claim 2 wherein the machine learning language model is a domain-invariant learning with bidirectional transformer for language understanding model.

5. The method of claim 2 wherein the machine learning language model is a dual intent and entity transformer.

6. The method of claim 1, further comprising, after receiving the first natural language input from the user:
using identifying information for the person to retrieve from an electronic medical record a list of prescriptions written for the person; and
providing third natural language output to the user identifying each of at least a portion of the prescriptions on the retrieved list.

7. The method of claim 1 wherein the discerned named entity is a drug name.

8. The method of claim 1, further comprising, after receiving the first natural language input from the user:
using identifying information for the user to retrieve from an electronic medical record, for each of a plurality of prescriptions written for the user, a drug name specified by the prescription; and
providing third natural language output to the user identifying each of at least a portion of the retrieved drug names.

9. The method of claim 1, further comprising, after receiving the second natural language input from the user:
using identifying information for the user and for the prescription to which the discerned named entity corresponds to retrieve from an electronic medical record program a record for the prescription to which the discerned named entity corresponds; and
determining from the retrieved record whether the prescription to which the discerned named entity corresponds is in condition to be refilled,
and wherein the causing a pharmacy refill order to be placed is performed in response to determining that the prescription to which the discerned named entity corresponds is in condition to be refilled.

10. The method of claim 1, further comprising, after receiving the second natural language input from the user:
using identifying information for the user and for the prescription to which the discerned named entity corresponds to retrieve from an electronic medical record a record for the prescription to which the discerned named entity corresponds;
determining from the retrieved record whether the prescription to which the discerned named entity corresponds is in condition to be refilled;
in response to determining that the prescription to which the discerned named entity corresponds is not in condition to be refilled, using identifying information in the retrieved record for a physician who wrote the prescription to which the discerned named entity corresponds to obtain authorization from the physician to amend the prescription to which the discerned named entity corresponds,
and wherein the causing a pharmacy refill order to be placed is performed in response to obtaining the authorization to amend.

11. The method of claim 1 wherein causing a pharmacy refill order to be placed comprises sending to a medical assistant associated with the prescription to which the discerned named entity corresponds a notification to order refill of the prescription from a pharmacy.

12. The method of claim 1 wherein causing a pharmacy refill order to be placed comprises calling a programmatic interface exposed by an electronic medical record to order refill of the prescription to which the discerned named entity corresponds.

13. The method of claim 1 wherein causing a pharmacy refill order to be placed comprises calling a programmatic interface exposed by a pharmacy to order refill of the prescription to which the discerned named entity corresponds.

14. The method of claim 1 wherein causing a pharmacy refill order to be placed comprises placing a telephone call to a telephone response system provided by a pharmacy to order refill of the prescription to which the discerned named entity corresponds.

15. One or more instances of computer-readable media collectively having contents configured to cause a computing system to perform a method, the method comprising:
conducting a conversational exchange with a person;
discerning from the exchange (1) an intent of refilling a prescription, and (2) an identification of a prescription to be fulfilled; and
in response to the discerning, causing a pharmacy refill order to be placed on behalf of the person for the identified prescription.

16. The one or more instances of computer-readable media of claim 15 wherein the conversational exchange is a spoken exchange in which the person's contributions are received via an audio input device, and in which the computing system's contributions are generated by a text-to-speech mechanism and outputted by an audio output device.

17. The one or more instances of computer-readable media of claim 15 wherein the conversational exchange is a free-text exchange in which the person's contributions are received via a text input device, and in which the computing system's contributions are outputted by a visual display device.

18. A method in a computing system, comprising:
conducting a conversational exchange with a person;
discerning from the exchange (1) an intent of refilling a prescription, and (2) an identification of a prescription to be fulfilled; and
in response to the discerning, causing a pharmacy refill order to be placed on behalf of the person for the identified prescription.
